SEQUENCE LISTING (12 sequences)

SEQ ID NO: 1 - DNA, 22 bp, synthetic construct
  source 1..22: mol_type = other DNA
ctggttgtccttgcagctgtag

SEQ ID NO: 2 - DNA, 26 bp, synthetic construct
  source 1..26: mol_type = other DNA
ccttttcaatctgagctcttctttct

SEQ ID NO: 3 - DNA, 20 bp, synthetic construct
  source 1..20: mol_type = other DNA
gtcagtggtggacctgacct

SEQ ID NO: 4 - DNA, 20 bp, synthetic construct
  source 1..20: mol_type = other DNA
aaaggtggaggagtgggtgt

SEQ ID NO: 5 - DNA, 21 bp, synthetic construct
  source 1..21: mol_type = other DNA
agatggatagccgagagtacc

SEQ ID NO: 6 - DNA, 23 bp, synthetic construct
  source 1..23: mol_type = other DNA
gcaaacctcatctcaaacacatc

SEQ ID NO: 7 - DNA, 20 bp, synthetic construct
  source 1..20: mol_type = other DNA
cccgctccactacaacgagt

SEQ ID NO: 8 - DNA, 20 bp, synthetic construct
  source 1..20: mol_type = other DNA
aacgcggtacatggtcctgt

SEQ ID NO: 9 - DNA, 22 bp, synthetic construct
  source 1..22: mol_type = other DNA
gttcaactgtgccagaggaaac

SEQ ID NO: 10 - DNA, 23 bp, synthetic construct
  source 1..23: mol_type = other DNA
tggtggaagaaagatagaaaccc

SEQ ID NO: 11 - DNA, 18 bp, synthetic construct
  source 1..18: mol_type = other DNA
gctcccattagtgccgtc

SEQ ID NO: 12 - DNA, 19 bp, synthetic construct
  source 1..19: mol_type = other DNA
ccctgctcctacacaacaa

(19) United States
(12) Patent Application Publication
(10) Pub. No.: US 2026/0092267 A1
(43) Pub. Date: Apr. 2, 2026

(54) FUSION PROTEIN COMPLEXES FOR USE IN EPIGENETIC REGULATION AND USE THEREOF

(71) Applicant: UNIVERSITY-INDUSTRY COOPERATION GROUP OF KYUNG HEE UNIVERSITY, Yongin-si (KR)

(72) Inventors: Seung Ja Oh, Yongin-si (KR); Na Ly Tran, Yongin-si (KR)

(21) Appl. No.: 19/070,685

(22) Filed: Mar. 5, 2025

(30) Foreign Application Priority Data
Mar. 6, 2024 (KR) 10-2024-0032056
Mar. 5, 2025 (KR) 10-2025-0028203

(51) Int. Cl. C12N 9/22; C12N 9/10; C12N 15/11

(52) U.S. Cl. CPC C12N 9/226; C12N 9/1007; C12N 15/11; C07K 2319/09; C12N 2310/20

(57) ABSTRACT

The disclosure relates to a fusion protein including a transcription enhancer and a dCas9 protein, a complex for epigenetic regulation including the fusion protein and one or more guide RNAs (gRNAs), etc.

The fusion protein or complex according to an aspect may induce apoptosis of colorectal cancer cells or compensate for rhodopsin deficiency by activating the gene EGFL8, opsin mw, etc., and may be utilized as a pharmaceutical composition or the like for preventing or treating cancer or ocular diseases.

### CROSS-REFERENCE TO RELATED APPLICATION

This application claims the benefit of Korean Patent Application No. 10-2024-0032056, filed on Mar. 6, 2024 and 10-2024-0028203, filed on Mar. 5, 2025, in the Korean Intellectual Property Office, the disclosures of which are incorporated herein in their entirety by reference.

### SEQUENCE LISTING

A sequence listing in XML format is filed herewith and is incorporated by reference in its entirety. The file is named PK078450US.xml, was created on Mar. 5, 2025, and has a size of 11,571 bytes.

### BACKGROUND

### 1. Field

The disclosure relates to a fusion protein including a transcription enhancer and a dCas9 protein, a complex for epigenetic regulation including the fusion protein and one or more guide RNAs (gRNAs), and the like.

### 2. Description of the Related Art

Cancer is a major cause of death worldwide, and the fight against cancer requires a multidirectional strategy to improve the effectiveness and accuracy of cancer treatment, but current treatment methods tend to focus on a single target of cancer cells.

Meanwhile, in addition to cancer cells, there are factors in the cancer microenvironment that support the progression of cancer, such as macrophages, and it is known that cancer cells directly or indirectly interact with macrophages and other cells in the cancer microenvironment. In addition to these factors, microbial metabolites, epigenetic modifications, etc., play a very important role in regulating communication between cancer cells and macrophages for cancer progression in the cancer microenvironment, but many aspects of communication between cancer cells and macrophages have not yet been clearly elucidated. Understanding the interactions and communication between cancer cells and macrophages in the tumor microenvironment is important for cancer research and will likely help in finding ways to treat cancer.

Against this background, the inventors of the disclosure sought to investigate a method of understanding the signaling relationship between cancer cells and macrophages through epigenetic regulation, and developed a novel dCas9-based histone methyltransferase system that is directly involved in the regulation of genetic expression in cancer cells. It is expected that the SETD7-dCas9 fusion protein according to an aspect and the complex including the same may be utilized as a pharmaceutical composition for preventing or treating cancer.

### SUMMARY

One aspect provides a fusion protein including a transcription enhancer and a deactivated Cas9 (dCas9) protein, wherein the fusion protein is characterized in that the transcription enhancer is SETD7 or a variant protein thereof.

Another aspect provides a polynucleotide encoding the fusion protein.

Another aspect provides a vector including the polynucleotide.

Another aspect provides a complex for epigenetic regulation, including the fusion protein and one or more guide RNAs (gRNAs), wherein the complex is characterized in that the transcription enhancer is SETD7 or a variant protein thereof.

Another aspect provides a composition for gene regulation including the complex.

Another aspect provides a pharmaceutical composition for preventing or treating cancer including the complex.

Another aspect provides a pharmaceutical composition for preventing or treating ocular diseases including the complex.

Another aspect provides an anti-aging composition including the complex.

Another aspect provides a method of treating cancer or an ocular disease, including administering the complex or a gene regulation composition including the complex to a subject.

Additional aspects will be set forth in part in the description which follows and, in part, will be apparent from the description, or may be learned by practice of the presented embodiments.

One aspect provides a fusion protein including a transcription enhancer and a deactivated Cas9 (dCas9) protein, wherein the transcription enhancer is SETD7 or a variant protein thereof.

It was confirmed that the SETD7-dCas9 fusion protein of the disclosure, according to an aspect, and a complex including the same may induce the death of colorectal cancer cells by activating the gene EGFL8, and may be utilized as a pharmaceutical composition, etc., for preventing or treating cancer.

In addition, it was confirmed that the SETD7-dCas9 fusion protein of the disclosure, according to an aspect, and a complex including the same may compensate for rhodopsin deficiency in cells by up-regulating the expression of the gene opsin mw, and may be utilized as a pharmaceutical composition, etc., for preventing or treating ocular diseases.

As used herein, the term “fusion protein” may refer to a protein formed by combining two or more originally separate proteins or portions thereof, and optionally may include a linker or spacer connecting the two or more proteins to each other.

As used herein, the term “transcription enhancer” may refer to a protein or a factor including the same that positively or negatively regulates the expression of a specific gene through DNA methylation, histone methylation/deacetylation, chromatin remodeling, etc. without epigenetically changing the DNA nucleotide sequence, and specifically may refer to a histone methyltransferase.

More specifically, the histone methyltransferase according to an aspect may be “SETD7” (SET7/9, KIAA1717, KMT7, SET7, SET9), which is a lysine methyltransferase (KMT) that methylates lysine at position 4 of histone H3 (H3K4). Transfer of methyl groups to lysine residues of different substrates is carried out by the Su (var) 3-9, Enhancer-ofzeste, and Trithorax (SET) domain of SETD7, which may enhance transcriptional activity by limiting condensation of chromatin and thereby monomethylation (H3K4me1). They are often located around enhancer binding sites and transcription start sites of transcribed genes.

In addition, the transcription enhancer may be characterized as being SETD7 or a variant protein thereof. The variant protein may refer to a protein in which one or more amino acids are conservatively substituted and/or modified, thereby differing from the amino acid sequence of the variant before the mutation, but maintaining the function or property, and the function or property of the variant protein may be increased, unchanged, or decreased compared to the polypeptide before the mutation. Specifically, the SETD7 variant protein according to an aspect may be, but is not particularly limited to, SETD7 H279A.

As used herein, the term “deactivated Cas9 (dCas9)” may refer to a Cas9 nuclease protein with deactivated nuclease function. The preparation of deactivated Cas9 protein according to an aspect may be prepared according to a usual method of deactivating the activity of nuclease, but is not particularly limited thereto.

The transcription enhancer and dCas9 protein may be connected via a linker, and the linker may consist of 10 to 15 amino acids in length. The linker according to an aspect is a linker that may enhance the binding affinity of dCas9 to the target gene, and may be a 15-acid amine linker, but is not particularly limited thereto.

The fusion protein may further include a nuclear localization signal (NLS) peptide in addition to the transcription enhancer and dCas9 protein. As used herein, the term “nuclear localization signal” refers to an amino acid sequence that functions to transport a specific substance (for example, a protein) into a cell nucleus, typically through a nuclear pore (Kalderon D, et al., Cell 39:499-509 (1984); Dingwall C, et al., J Cell Biol. 107 (3): 8419 (1988)). Specifically, the nuclear localization signal according to an aspect may bind to the C-terminus of the dCas9 protein.

Another aspect provides a polynucleotide encoding the fusion protein. The same portion as described above also applies to the polynucleotide.

As used herein, the term “polynucleotide” may refer to a polymer of deoxyribonucleotides or ribonucleotides present in single-stranded or double-stranded form. This includes RNA genomic sequences, DNA (gDNA and cDNA) and RNA sequences transcribed therefrom, and unless otherwise specified, includes analogs of natural polynucleotides.

In the disclosure, not only the nucleotide sequence of the polynucleotide encoding the fusion protein, but also the nucleotide sequence encoding a protein that exhibits substantially the same or corresponding efficacy as each of the above proteins as the nucleotide sequence showing 80% or more, specifically 90% or more, more specifically 95% or more, even more specifically 98% or more, and most specifically 99% or more homology with the above sequence are included in the scope of the disclosure without limitation. In addition, it is obvious that even if a sequence has substantially the same or corresponding biological activity as the sequence having homology to the above sequence, a case in which part of the sequence is deleted, modified, substituted or added is also included in the scope of the disclosure. In addition, the polynucleotide also includes a polynucleotide sequence encoding the fusion protein as well as a sequence complementary to that sequence.

Another aspect provides a vector including the polynucleotide. The same aspects as described above apply equally to the vector.

As used herein, the term “vector” may refer to a recombinant vector capable of expressing a target protein when introduced into an appropriate host cell, and may refer to a genetic construct including essential regulatory elements operably connected to enable expression of a gene insert, and may be used interchangeably with the term “expression vector.”

An expression vector according to an aspect may include, in addition to expression regulatory factors such as a promoter, an initiation codon, a termination codon, a polyadenylation signal, and an enhancer, a signal sequence for membrane targeting or secretion, and may further include a selection marker for selecting a host cell containing the vector.

Another aspect provides a complex for epigenetic regulation, including the fusion protein and one or more guide RNAs (gRNAs), wherein the complex is characterized in that the transcription enhancer is SETD7 or a variant protein thereof. The same aspects as described above apply equally to the complex.

As used herein, the term “guide RNA (gRNA)” refers to a small RNA of about 45 to 70 nucleotides having nucleotide sequence information that serves as a template for a modification reaction when editing RNA, and according to an aspect, the guide RNA may be an sgRNA that targets a gene promoter at various locations upstream of a transcription start site (TSS), but is not particularly limited thereto.

Another aspect provides a composition for gene regulation including the complex. The same aspects as described above apply equally to the composition.

Specifically, the gene may be a cancer-related gene, and the cancer-related gene may be any one or more selected from the group consisting of EGFL8, HLA-G, BRD3, FNTB, and ERCC5, and more specifically, may be EGFL8, but is not particularly limited thereto. EGFL8 (EGF like domain multiple 8) is a colorectal cancer-related gene that shows low expression in colorectal cancer patients, and therefore, up-regulation of EGFL8 may induce apoptosis in cancer.

In an experimental example, SETD7-dCas9 and EGFL8 sgRNA were transfected into colorectal cancer cells HCT-116, and it was confirmed that SETD7-dCas9 may enhance the expression of EGFL8 in HCT-116 (FIG. 5A). In addition, it was confirmed from AnexinV/7AA data that SETD7-dCas9 may induce apoptosis in SETD7-dCas9-transfected HCT-116 cells by enhancing EGFL8 (FIG. 5B, C).

Furthermore, the gene may be any one or more selected from the group consisting of opsin mw, STK33, and TNUK. In an experimental example, it was confirmed that the expression of the opsin mw gene may be up-regulated by SETD7-dCas9 including gRNA (FIG. 4D).

The gene regulation composition may effectively regulate target DNA of eukaryotic cells ex vivo or in vivo. For example, it may be cultured cells (in vitro), transplanted cells (graft cells) and primary cell cultures (in vitro and ex vivo), and in vivo cells, cells of an organism or mammalian cells, including human cells, which are commonly used in the art.

Another aspect provides a pharmaceutical composition for preventing or treating cancer including the complex. The same aspects as described above apply equally to the pharmaceutical composition.

As used herein, the term “cancer” may refer to a tumor that has grown abnormally due to autonomous excessive growth of body tissue, or a disease that forms a tumor. Specifically, the cancer may be any one or more selected from the group consisting of colorectal cancer, liver cancer, lung cancer, pancreatic cancer, non-small cell lung cancer, colon cancer, bone cancer, skin cancer, head or neck cancer, cutaneous or intraocular melanoma, uterine cancer, ovarian cancer, rectal cancer, gastric cancer, anal cancer, colon cancer, breast cancer, fallopian tube carcinoma, endometrial carcinoma, cervical carcinoma, vaginal carcinoma, vulvar carcinoma, Hodgkin's disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, bladder cancer, kidney or ureteral cancer, renal cell carcinoma, renal pelvic carcinoma, central nervous system (CNS) tumor, primary central nervous system lymphoma, spinal cord tumor, brainstem glioma, and pituitary adenoma, and more specifically, may be colorectal cancer, but is not particularly limited thereto.

Another aspect provides a pharmaceutical composition for preventing or treating ocular diseases including the complex. The same aspects as described above apply equally to the pharmaceutical composition.

Specifically, the ocular disease may be any one or more selected from the group consisting of vision impairment/loss, presbyopia, diabetic retinopathy, blepharitis, retinitis, keratitis, conjunctivitis, optic neuritis, dry eye syndrome, glaucoma, cataract, and macular degeneration, and more specifically, may be vision impairment/loss, but is not particularly limited thereto. As used herein, the term “prevention” refers to any act by which the onset of a cancer disease or disease possibility is inhibited or delayed by administration of the composition of the disclosure, and the term “treatment” refers to any act by which the symptoms of a cancer disease are improved or beneficially changed by administration of the composition of the disclosure.

The pharmaceutical composition may include a pharmaceutically acceptable carrier. The term “pharmaceutically acceptable carrier” may refer to a carrier or diluent that does not irritate the organism and does not interfere with the biological activity and properties of the compound being administered. In this context, “pharmaceutically acceptable” refers to not inhibiting the activity of the active ingredient and does not have more than adaptable toxicity to the subject to whom it is applied (prescribed).

The type of carrier that may be used in the disclosure may be any carrier commonly used and pharmaceutically acceptable in the art. Non-limiting examples of the above carriers may include saline solution, sterile water, Ringer's solution, buffered saline, albumin injection solution, dextrose solution, maltodextrin solution, glycerol, ethanol, etc. These may be used either individually or in combination with two or more types. The pharmaceutical composition may be prepared as an oral dosage form or a parenteral formulation, depending on the route of administration, by common methods known in the art, including pharmaceutically acceptable carriers in addition to the active ingredient.

The pharmaceutical compositions may be prepared and used according to common methods in the form of oral dosage forms, topical preparations, suppositories, or sterile injectable solutions, such as pills, granules, tablets, capsules, suspensions, emulsions, syrups, aerosols, etc. The pharmaceutical compositions may be prepared by adding diluents or excipients such as commonly used fillers, extenders, binders, lubricants, disintegrants, or surfactants, etc.

When the pharmaceutical composition is formulated as an oral dosage form, the pharmaceutical composition may be prepared as a powder, granule, tablet, pill, disintegrating tablet, capsule, liquid, gel, syrup, suspension, wafer, etc., in combination with a suitable carrier, according to methods known in the art. Examples of suitable pharmaceutically acceptable carriers include sugars such as lactose, glucose, sucrose, dextrose, sorbitol, mannitol, and xylitol, etc., starches such as corn starch, potato starch, and wheat starch, etc., celluloses such as cellulose, methylcellulose, and ethylcellulose, sodium carboxymethylcellulose and hydroxypropylmethylcellulose, etc., polyvinyl pyrrolidone, water, methylhydroxybenzoate, propylhydroxybenzoate, magnesium stearate, mineral oils, malt, gelatin, talc, polyols, vegetable oils, etc. In the case of formulation, if necessary, the pharmaceutical composition may be prepared to include diluents and/or excipients such as fillers, extenders, binders, lubricants, disintegrants, surfactants, etc.

When the pharmaceutical composition is formulated as a parenteral formulation, the pharmaceutical composition may be prepared in the form of an injectable formulation, transdermal delivery system, nasal inhaler, and suppository according to methods known in the art in combination with a suitable carrier. When formulated as an injectable formulation, sterile water, ethanol, polyols such as glycerol or propylene glycol, etc., or mixtures thereof, may be used as suitable carriers, desirably isotonic solutions such as Ringer's solution, phosphate buffered saline (PBS) containing triethanolamine, sterile injectable water, 5% dextrose, etc., may be used. When formulated as a transdermal delivery system, the pharmaceutical composition may be prepared as an ointment, a cream, a lotion, a gel, a topical solution, a paste, a liniment, or an aerosol, etc. In the case of nasal inhalers, the pharmaceutical composition may be prepared as an aerosol spray using a suitable propellant such as dichlorofluoromethane, trichlorofluoromethane, dichlorotetrafluoroethane, carbon dioxide, etc., when formulated as a suppository, witepsol, tween 61, polyethylene glycol, cacao glycol, laurin glycol, polyoxyethylene sorbitan fatty acid ester, polyoxyethylene stearate, sorbitan fatty acid ester, etc., may be used as the base.

The pharmaceutical composition may be administered in a pharmaceutically effective amount, wherein the term “pharmaceutically effective amount” refers to an amount sufficient to treat or prevent a disease with a reasonable benefit/risk ratio applicable to medical treatment or prevention, and an effective dose level may be determined by factors including severity of the disease, activity of a drug, age, weight, health, and sex of a patient, sensitivity of the patient to a drug, time of administration, route of administration, and elimination rate of the composition of the disclosure used, duration of treatment, the drugs used in combination or concurrently with the composition of the disclosure, and other factors well known in the medical field. The pharmaceutical composition may be administered alone or in combination with components known to exhibit therapeutic effects against cancer. It is important to administer an amount that may obtain the maximum effect with the minimum amount without side effects in consideration of all the above factors.

The dosage of the pharmaceutical composition may be determined by those skilled in the art, taking into account the intended use, the severity of the disease, the patient's age, weight, gender, pre-existing conditions, or the type of substance used as an active ingredient, etc. For example, the pharmaceutical compositions of the disclosure may be administered at a dose of from about 0.1 ng to about 1,000 mg/kg per adult, desirably from 1 ng to about 100 mg/kg, and the frequency of administration of the compositions of the disclosure is not particularly limited, but may be once daily or multiple times in divided doses. The above dosage or frequency of administration is not intended to limit the scope of the disclosure in any way.

Another aspect provides an anti-aging composition including the complex. The same aspects as described above apply equally to the composition.

The anti-aging composition may be an anti-aging cosmetic composition or a topical preparation composition, and as used herein, the term “anti-aging” in the specification may refer to any action that inhibits or alleviates the progression of aging.

Another aspect provides a method of treating cancer or an ocular disease, including administering the complex or a gene regulation composition including the complex to a subject. The same aspects as described above apply equally to the method.

A method of treating cancer or an ocular disease according to an aspect includes administering to a subject the complex or a gene regulating composition including the complex, thereby not only treating the disease itself before the onset of symptoms, but also inhibiting or avoiding symptoms thereof. In the management of a disease, the prophylactic or therapeutic dosage of a particular active ingredient may vary depending on the nature and severity of the disease or condition and the route by which the active ingredient is administered.

The dosage and frequency of administration of the complex or the gene regulating composition including the complex may vary depending on the age, body weight and response of each individual patient, and an appropriate dosage regimen may be readily selected by a person of ordinary skill in the art who naturally takes these factors into consideration.

Furthermore, the method of treating the cancer or ocular disease may further include administering a therapeutically effective amount of an additional active agent helpful in treating the disease together with the complex or the gene regulating composition including the complex, wherein the additional active agent may exhibit a synergistic effect or auxiliary effect together with the complex or the gene regulating composition including the complex.

### BRIEF DESCRIPTION OF THE DRAWINGS

These and/or other aspects will become apparent and more readily appreciated from the following description of the embodiments, taken in conjunction with the accompanying drawings in which:

FIGS. 1A to 1D relate to a preparation of a CRISPR/dCas9-based histone methyltransferase SETD7-dCas9 used in the disclosure. FIG. 1A schematically illustrates the structure of the SETD7-dCas9 system, and FIG. 1B illustrates different structures of SETD7-dCas9 including SETD7 wild type (WT), SETD WT mutant H297A, SETD7 C-term, and SETD7 C-term mutant H297A-based dCas9, respectively. FIG. 1C illustrates a simulation structure of SETD7-dCas9, and FIG. 1D illustrates the results of measuring the expression of SETD7-dCas9 proteins of different structures by Western blot (SETD7-dCas9: 204 kDa, SETD7 cterm-dCas9: 180 kDa, and dCas9: 160 kDa).

FIGS. 2A to 2C relate to SETD7-dCas9 protein purification and functional confirmation, wherein FIG. 2A illustrates a simulation of the SETD7-dCas9 protein structure, FIG. 2B illustrates in vitro H3K4 methylation activity, and FIG. 2C illustrates a simulation of binding between Histone H3 and SETD7-dCas9.

FIGS. 3A to 3D relate to the activation of SETD7 target genes by SETD7-dCas9, wherein FIG. 3A illustrates the results of measuring differentially expressed genes in SET7 knockdown compared to wild type using RNA-seq, FIG. 3B illustrates SETD7-dCas9-activated mRNA expression of natural target genes, and FIGS. 3C and 3D illustrate mRNA HLA-G expression using various SETD7-dCas9 constructs.

FIGS. 4A to 4D relate to H3K4 methylation of SETD7-dCas9 at the transcription start site of HLA-G and the activation of other target genes, wherein FIG. 4A illustrates chromatin fragmented from 300 bp to 800 bp by sonication, and FIG. 4B illustrates the results of H3K4met1 ChIP qPCR at the TSS of HLA-G, HLA-G gene body, and FNTB gene body. FIG. 4C and FIG. 4D illustrate the activation of the target OCT4 gene, opsin mw, and RORα genes by SETD7-dCas9, respectively.

FIG. 5A illustrates the results of measuring EGFL8 activation of SETD7-dCas9 and p300-dCas9 by RT-PCR, and FIG. 5B illustrates the results of measuring enhanced apoptosis by SETD7-dCas9 and p300-dCas9 by AnexinV/7AAD flow cytometry.

FIG. 6A illustrates the results of measuring the viability of SETD7-dCas9 and p300-dCas9 transfected spheroids by Cell Titer Glow, and FIG. 6B illustrates the results of measuring SETD7-dCas9 and p300-dCas9-induced macrophage polarization by DC-SIGN (M1 marker) and HLA-DR (M2 marker) flow cytometry.

FIG. 7 illustrates a predictive model for the activity of SETD7-dCas9;

FIG. 8 illustrates a box plot showing the median, quartiles and range of DNA methylation values of promoter CpGs for each cell line of nine genes (BRD3, EGFL8, HLA-G, TP53, FNTB, ERCC5, VEGFA, CDKN1A, MDM2) and the optimal threshold values obtained using the Youden index.

FIG. 9 illustrates a heat map of differentially methylated promoter CpG sites for 12 genes (HLA-G, BRD3, EGFL8, TP53, OCT4, OPN1MW, RORA, FNTB, ERCC5, VEGFA, CDKN1A, MDM2) selected from 748 different cell lines, with red indicating high methylation status and blue indicating low methylation status.

FIG. 10 illustrates expression of MW opsin mRNA in NIH-3T3 cells after transfection with SETD7-dCas9 and MW opsin-specific gRNA.

FIG. 11 illustrates expression of MW opsin protein in NIH-3T3 cells after transfection with SETD7-dCas9 and MW opsin-specific gRNA.

FIG. 12 is a schematic representation of the light absorption properties of NIH-3T3 cells expressing MW opsin activated by SETD7-dCas9 and treated with 11-cis-retinal.

FIG. 13 illustrates changes in the absorption spectrum of NIH-3T3 cells manipulated with SETD7-dCas9.

FIG. 14 illustrates light absorption in NIH-3T3 cells expressing rhodopsin.

FIG. 15 illustrates the results of a comparative light absorption analysis showing that MW opsin may compensate for rhodopsin deficiency.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein.

Accordingly, the embodiments are merely described below, by referring to the figures, to explain aspects of the present description. As used herein, the term “and/or” includes any and all combinations of one or more of the associated listed items.

### Experimental Example 1: Material and Method

### 1-1. Plasmid Preparation

To clone SETD7-dCas9, a mutagenesis kit was used to prepare dCas9 from the pET-Cas9-NLS-6×His vector (Addgene). Afterwards, SETD7 was connected to dCas9 using a linker that may improve the binding affinity of dCas9 to the target gene. SETD7 was bonded to dCas9 via NEBuilder HiFi DNA Assembly (NEB, E2621) and cloned into pCDNA3 and pET backbone plasmids. pCDNA3-SETD7 H279A-dCas9, pCDNA3-SETD7 Cterm-dCas9, pCDNA3 SETD7 H279A Cterm-dCas9, and pCDNA3 SETD7 Nterm-dCas9 were cloned using NEBuilder HiFi DNA assembly (NEB, E2621), and all enzymes were purchased from New England Biolabs.

### 1-2. Purification of Cas9 Fusion Protein

A pET-SETD7-dCas9-NLS-6×His plasmid was transformed into an Escherichia coli BL21 (DE3) competent cell and cultured overnight at 37° C. using Luria-Bertani agar plates including ampicillin (100 μg/ml). Transfected BL21 cells were selected and cultured overnight at 20° C. and 120 rpm in 3 L of LB-ampicillin broth including 1 mM isopropyl-β-D-thiogalactopyranoside (IPTG). Cells were collected by ultracentrifugation and lysed in lysis buffer (50 mM NaH2PO4, 300 mM NaCl, 5 mM imidazole (pH 8.0)). After ultracentrifugation at 18,000 rpm for 40 minutes at 4° C., the soluble lysate was cultured on Ni-NTA resin (Thermo Fisher Scientific) at 4° C. for 2 hours. After washing with wash buffer (50 mM NaH2PO4, 300 mM NaCl, 20 mM imidazole) (pH 8.0), the solution was eluted with elution buffer (50 mM NaH2PO4, 300 mM NaCl, 100 mM imidazole (pH 8.0), 50 mM NaH2PO4, 300 mM NaCl, 250 mM imidazole (pH 8.0)). After concentration, the buffer was replaced with PBS, and the purity of the SETD7-Cas9 protein was confirmed by Coomassie blue SDS-PAGE gel staining.

### 1-3. Complex Structure Modeling

A three-dimensional structure of Cas9 was modeled using the Galaxy Loop method based on a template structure (PDB ID: 6IFO). A single-stranded structure of mRNA1X and mRNA3X were modeled using the Rosetta software suite. A 3D structure was generated using the Rna_de_novo program of the Rosetta suite based on a predicted secondary structure of the RNA strands from the RNAFold server. All measurements of the protein structure were described by ChimeraX.

1-4. mRNA Transcriptome Sequencing

HEK293 cells were knocked down by 50 ng of SETD7 siRNA. Afterwards, total mRNA was extracted from the knockdown cells using an RNeasyR Mini Kit (QIAGEN, Cat #74104). Samples were analyzed with a TruSeq Stranded mRNA LT Sample Preparation Kit using an Illumina platform for next-generation sequencing (Macrogen). The experimental flow was based on Nat Rev Genet (2011 Sep. 7; 12 (10): 671-82). The data were analyzed by Macrogen, and genes with an average q value less than 0.05 were considered as differentially expressed targets between wild type and SETD7 siRNA knockdown cells. Genes were selected as candidates if the fold change was 4 or more and the read count value was 100 or more.

1-5. gRNA Design and Synthesis

gRNA sequencing was designed by the Crispr-era.stanford.edu website or the following references. sgRNAs targeting gene promoters at various locations upstream of the transcription start site (TSS). The 20 bp binding site sequence analysis of sgRNA targeting a specific gene was cloned into pRG2 by Bsal restriction enzyme ligation.

### 1-6. ChIP-qPCR Enrichment Assay

8.4 μg of pCDNA3-dCas9-6His or pCDNA3-SETD7-dCas9-6His and 2.4 μg of sgRNA were co-transfected into 7×10\hat{ }6 HEK293 cells in 10 cm plates with 15 μl Lipofectamin 2000. Harvested cells were cross-linked with 11% fixation buffer and suspended in glycine at a 1:10 ratio. Chromatin was sheared on ice for 25 minutes using Bioruptor KR (CosmoBio Co., Ltd., Tokyo, Japan) with 15 seconds of sonication at 30 second intervals to obtain DNA fragments with an average length of about 100-500 bp. ChIP qPCR enrichment analysis was performed according to the ChIP Kit Magnetic qPCR (ab270816). The sheared chromatin was cultured overnight together with antibodies previously bound to magnetic bead antibodies to H3K4me1 (EMD Millipore 07-473), H3K27ac (EMD Millipore 07-360), and IgG. After reverse cross-linking and DNA isolation, primers were designed to amplify a region of ˜324 to ˜49 bps spanning a HLA-G binding motif within a gene promoter region. qChIP primer sequences are shown in Table 1 below.

TABLE 1

| | | | Size |
|---|---|---|---|
| Genes | Sequence (5′-3′) | | (bp) |
| hHLA-G | Forward | CTGGTTGTCCTTGCAGCTGTAG (SEQ ID NO: 1) | 80 |
| | Reverse | CCTTTTCAATCTGAGCTCTTCTTTCT (SEQ ID NO: 2) | |
| hGAPDH | Forward | GTCAGTGGTGGACCTGACCT (SEQ ID NO: 3) | 166 |
| | Reverse | AAAGGTGGAGGAGTGGGTGT (SEQ ID NO: 4) | |
| hBRD3 | Forward | AGATGGATAGCCGAGAGTACC (SEQ ID NO: 5) | 147 |
| | Reverse | GCAAACCTCATCTCAAACACATC (SEQ ID NO: 6) | |
| hEGFL8 | Forward | CCCGCTCCACTACAACGAGT (SEQ ID NO: 7) | 106 |
| | Reverse | AACGCGGTACATGGTCCTGT (SEQ ID NO: 8) | |
| hFNTB | Forward | GTTCAACTGTGCCAGAGGAAAC (SEQ ID NO: 9) | 146 |
| | Reverse | TGGTGGAAGAAAGATAGAAACCC (SEQ ID NO: 10) | |
| hERCC5 | Forward | GCTCCCATTAGTGCCGTC (SEQ ID NO: 11) | 77 |
| | Reverse | CCCTGCTCCTACACAACAA (SEQ ID NO: 12) | |

### 1-7. In Vitro Histone H3 (K4) Methyltransferase Assay

The H3K4 methyltransferase activity of a SETD7-dCas9 protein was measured using a Histone H3 (K4) Methyltransferase Activity Quantitative Assay Kit (ab 113452).

### 1-8. RT-PCR

Total RNA from macrophages was extracted using the RNeasyR Mini Kit (QIAGEN, Cat #74104). 2.5 μg of total cellular RNA was converted to cDNA using the SuperScript™ VILO™ Master Mix Kit for cDNA Synthesis (Invitrogen™, Cat #11755250). Primers were purchased from Bioneer (Daejeon), and real-time PCR was performed using diluted cDNA using an Applied Biosystems model 7500 real-time cycler, and the relative level of mRNA gene expression was analyzed using the 2-ΔΔCT method.

### 1-9. Apoptosis Measurement by Annexin V/7-AAD Flow Cytometry

HCT116 cells were seeded at 0.35×106 per well in a 6-well plate. After 12 hours, HCT116 cells were transfected with 8.4 μg of SETD7-dCas9 and 2.8 μg of gRNA including Lipofectamin 2000 and cultured for 48 hours. After isolating cells with trypsin, centrifugation was performed at 2000 rpm for 2 minutes and the supernatant was removed. The Cells were washed once with cold FACS buffer (500 mL PBS, 5 mL FBS, 5 mL P/s, 1 mM EDTA) and suspended in 1X binding buffer (BD). 5 μl of APC Annexin V (BD, Cat #5504774) and 2 μl of 7-AAD (BD, Cat #51-68981E) were added to the cells and cultured at room temperature (25° C.) in the dark for 15 minutes. 1X Binding Buffer was added to each tube, and the stained cells were analyzed by flow cytometry within 1 hour. Samples included unstained cells, cells stained with APC Annexin V alone (no 7-AAD), and cells stained with 7-AAD alone (no APC Annexin V) to establish compensation, and data were analyzed with FlowJo software.

### 1-10. Human Cell Line

Human cancer cell lines MDA-MB-231, HCT116, and human monocyte cell line THP-1 (TIB-202™) cells were purchased from the Korea Cell Line Bank. Cell lines MDA-MB-231, HCT116, and THP-1 were maintained in Roswell Park Memorial Institute (RPMI) medium supplemented with 10% FBS and antibiotics (100 U/ml penicillin, 100 μg/ml streptomycin) from Gibco (Carlsbad, CA, USA). HEK-293T was purchased from the Korean Cell Line Bank and cultured in DMEM medium supplemented with 10% FBS and antibiotics (100 U/ml penicillin, 100 μg/ml streptomycin) from Gibco (Carlsbad, CA, USA).

### 1-11. Statistical Analysis

All experiments were performed three times, and all statistical values are expressed as mean±standard deviation (SD) or ±standard error (SE). Furthermore, student's t-test was used to verify the statistical significance of the results, and a p-value less than 0.05 was considered significant (* P<0.05, ** P<0.01, *** P<0.001).

### Experimental Example 2: Experimental Results

### 2-1. CRISPR/dCas9-Based Histone Methyltransferase

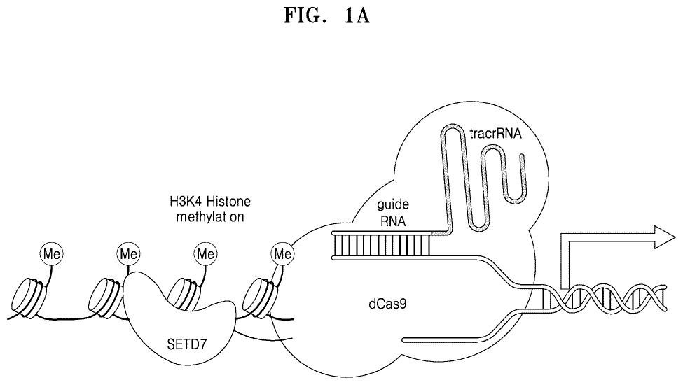

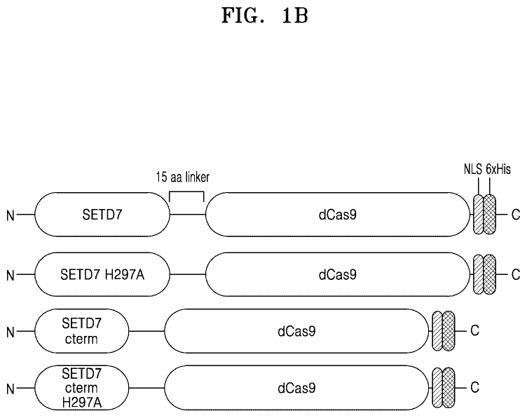

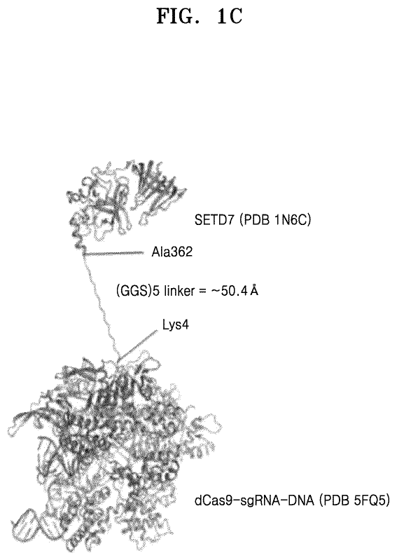

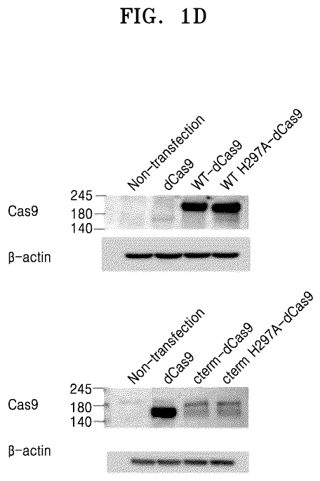

To understand epigenetic regulation of gene expression in cancer, a CRISPR/dCas9-based histone methyltransferase was first prepared. The system includes two parts, SETD7 located at an N-terminus of a protein and dCas9 located at a C-terminus. After dCas9 including gRNA binds to a transcription start site (TSS) region of the target gene, SETD7 binds to and methylates histone H3 (FIG. 1A). The system was developed with four constructs, the first of which is wild type SETD7 connected to dCas9 using a 15-acid amine linker to enhance binding efficiency. The following construct is a SETD7 H297A mutant-dCas9 that abolishes SETD7 methylase activity. The SETD7 C-term includes only a regulatory portion of the enzyme and was merged with dCas9 to compare its activity with that of wild type SETD7. Finally, SETD7 C-term H297A mutation-dCas9 was prepared to confirm the decrease of the enzyme after the enzyme activity disappeared (FIG. 1B). A simulation model of SETD7-dCas9, in which the linker and sgRNA bind to dCas9, was also established (FIG. 1C). FIG. 1D illustrates that the four constructs may express proteins after being transfected into HEK293 cells.

### 2-2. SETD7-CRISPR/dCas9 Structural Analysis and Activity

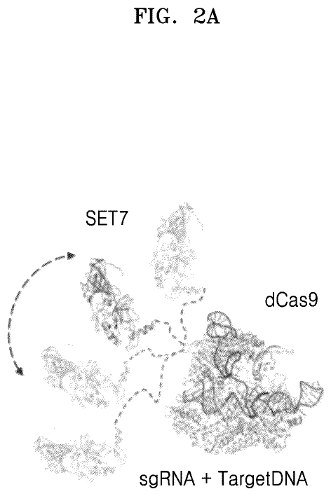

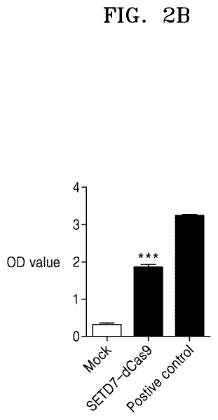

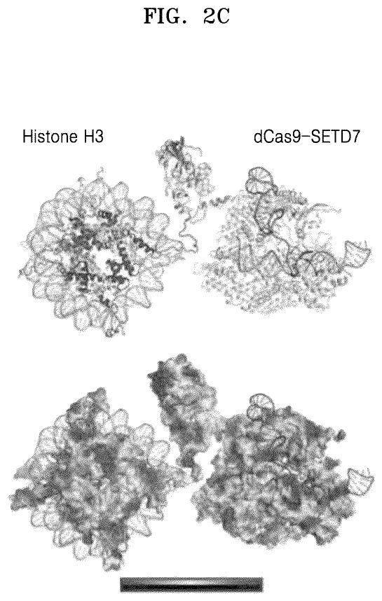

After establishing the SETD-dCas9 system, the H3K4 methylation ability was confirmed through a simulation model and protein activity. FIG. 2A illustrates that using a linker between dCas9 and SETD7 may make SETD7 flexible. A simulation model of histone H3 and SETD7-dCas9 showed the binding between SETD7 and histone H3 (FIG. 2C). In addition, the SETD7-dCas9 protein was confirmed to have H3K4 methylation activity with H3K4 histone substrate through in vitro analysis (FIG. 2B).

### 2-3. Confirmation of SETD7-dCas9 Activation of SETD7 Target Gene

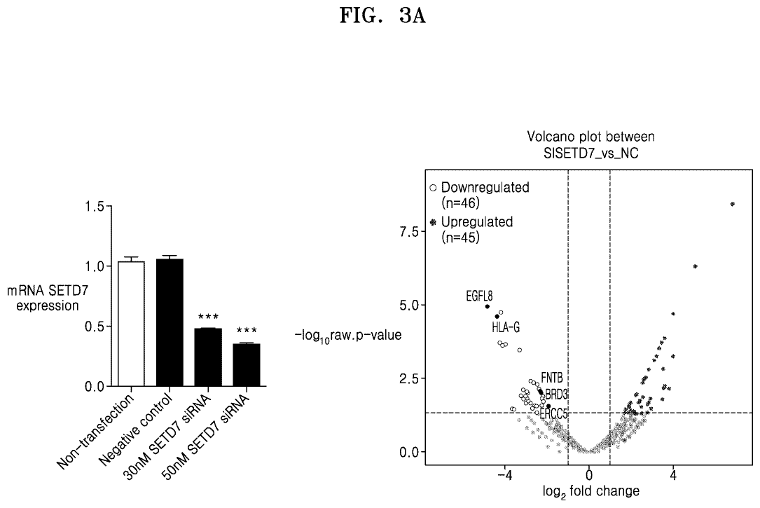

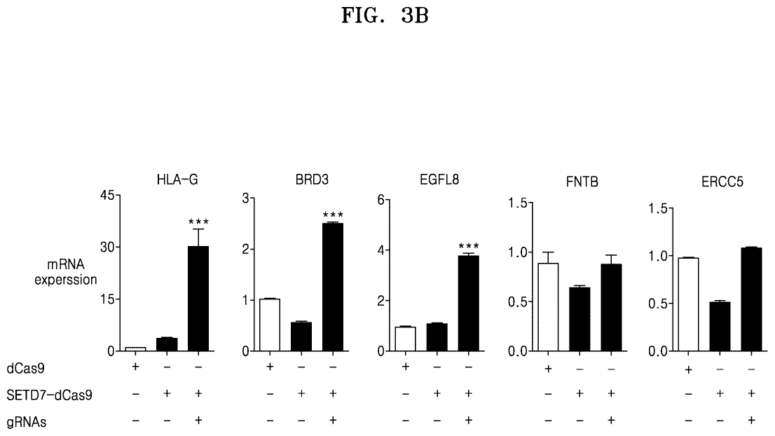

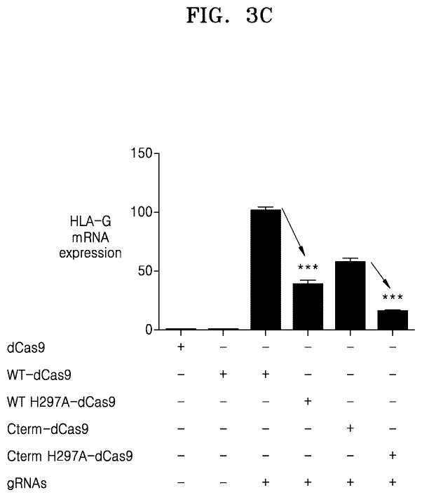

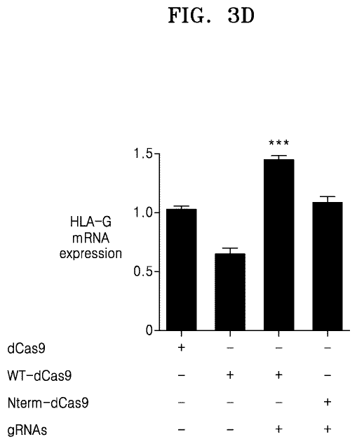

To test whether the SETD7-dCas9 fusion protein may regulate endogenous histone methylation or gene expression, comparative RNA-seq was first used to identify natural targets of SETD7. A SETD7 knockdown was generated in HEK293 cells. Knockdown SETD7 transcriptome-wide changes were compared to wild type HEK293 cells. In addition, comparative RNA-seq between WT and SETD7 knockdown cells generated 45 down-regulated genes and 46 up-regulated genes, respectively (FIG. 3A).

Meanwhile, guide RNAs (gRNAs) were designed to recruit each fusion protein before the transcription start site (TSS) of the top five most significantly down-regulated genes (HLA-G, BRD3, EGFL8, FNTB, and ERCC5). As a result, SETD7-dCas9 including gRNA showed activation of HLA-G, BRD3, and EGFL8, but not FNTB and ERCC5 (FIG. 3B). FNTB and ERCC5 may not be direct targets of SETD7 and therefore cannot be enhanced by the SETD-dCas9 system.

Next, the effectiveness of various SETD7-dCas9 constructs in gene regulation was investigated. Four different constructs of SETD7-dCas9 and gRNA of the HLA-G gene were transfected into HEK293 cells, and mRNA expression was measured by RT-PCR. As a result, wild type (WT) SETD7-dCas9 was shown to have the highest efficacy in HLA-G gene expression only for the enzyme regulatory portion SETD7 C-term-dCas9 (FIG. 3C). When both WT SETD7 and the C-term fusion platform were point-mutated to H297A to eliminate methyltransferase activity, HLA-G mRNA expression was also reduced. That is, WT SETD7-dCas9 was confirmed to be the most efficacious among the four constructs. To reaffirm this, SETD7 N-term-dCas9 was prepared and co-transfected with gRNA of HLA-G, then compared with WT SETD7-dCas9. As a result, it was confirmed that SETD7 N-term-dCas9 cannot activate HLA-G mRNA expression.

### 2-4. SETD7-CRISPR/dCas9 H3K4met1 in Transcription Start Site (TSS) Enrichment Confirmation

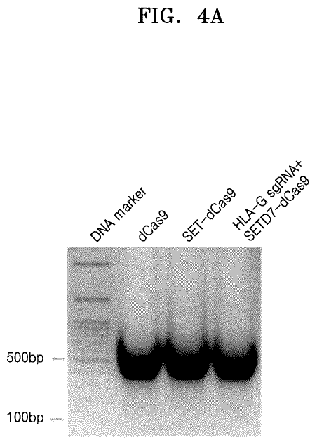

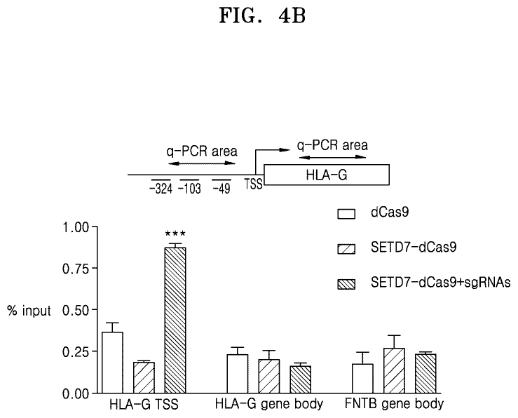

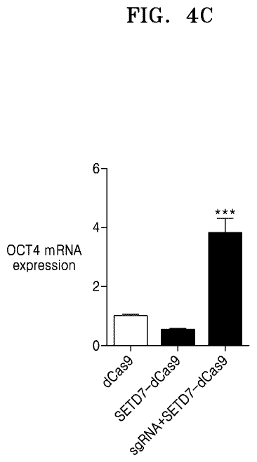

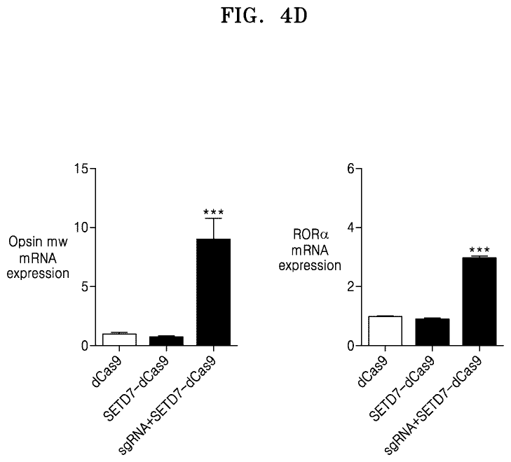

Since SETD7 is known as an H3K4met1 histone methyltransferase, to confirm whether gene expression activation is associated with methylation activity, three gRNAs targeting HLA-G at the transcription start site were transfected together with SETD7-dCas9 in HEK293. Subsequently, chromatin was fragmented to 300 bp to 800 bp through sonication (FIG. 4A). As a result, H3K4met1 Chip qPCR analysis showed that co-transfection of gRNA and SETD7-dCas9 may methylate H3K4 at the TSS region, whereas the HLA-G gene body region or the TSS location of another gene FNTB did not show methylation activity (FIG. 4B).

2-5. Confirmation of Activation of Other Target Genes by SETD7-dCas9

To confirm the effect of SETD7-dCas9 on non-natural SETD7 target genes, OCT4 was selected as a common target commonly used to evaluate other dCas9 fusion activation systems. As a result, it was confirmed that co-transfection of SETD7-dCas9 with gRNA may activate OCT4 activation (FIG. 4C). Next, the activation of SETD7-dCas9 was confirmed in the opsin and RORα genes. To transfect three gRNAs of opsin-mw, SETD7-dCas9 was used in NIH-3T3 cells and RORα was used in HEK-293 cells. As a result, it was confirmed that SETD7-dCas9 including gRNA may up-regulate opsin mw and RORα gene expression (FIG. 4D).

### 2-6. SETD7-CRISPR/dCas9 Activation of Tumor Regulatory Genes for Cancer Therapy in Colorectal Cancer Cell Line HCT-116

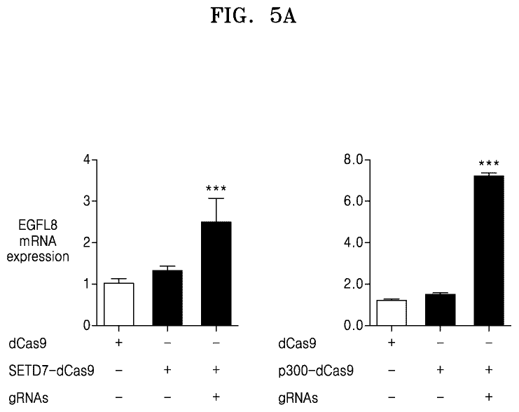

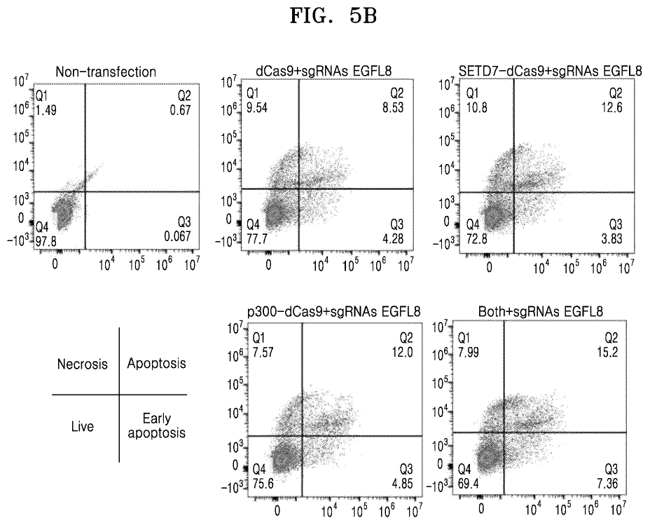

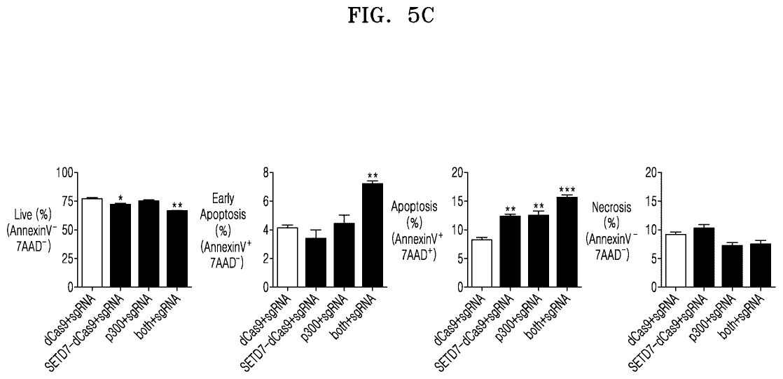

After confirming the activation of SETD7-dCas9 in various genes in HEK-293 cells, it was confirmed whether this system regulates cancer regulatory genes in the colorectal cancer cell line HCT-116. EGFL8 is a gene associated with colorectal cancer and shows low expression in colorectal cancer patients, therefore, up-regulation of EGFL8 may induce apoptosis in cancer. Specifically, an attempt was made to transfect SETD7-dCas9 and EGFL8 sgRNA in HCT-116, and also attempted to use p300-dCas9, another acetylated epigenetic enzyme well known as an upstream regulator. As a result, it was shown that both SETD7-dCas9 and p300-dCas9 may enhance the expression of EGFL8 in HCT-116 (FIG. 5A).

Next, whether the expression of EGFL8 may induce apoptosis in colorectal cancer was investigated. As a result, AnexinV/7AA data indicated that transfected SETD7-dCas9 and p300 may enhance EGFL8 and induce apoptosis in HCT-116. Furthermore, it was confirmed that co-transfection of SETD7-dCas9 and p300-dCas9 may induce more apoptosis than single transfection (FIG. 5B, C).

### 2-7. Cancer-Macrophage Interactions Via Epigenetic Modification of 3D Spheroids

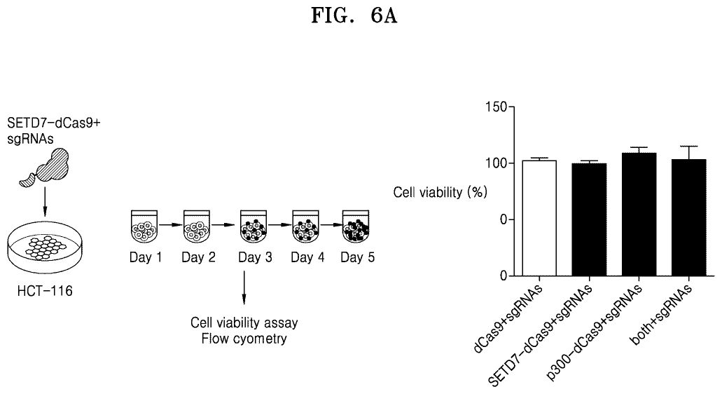

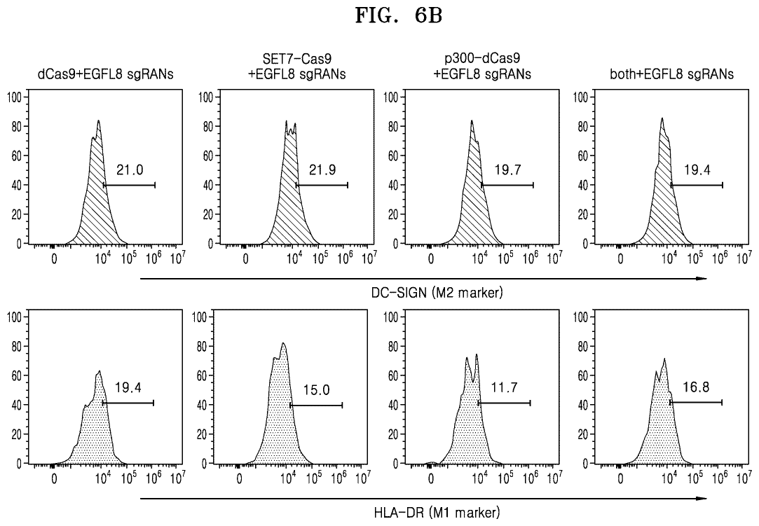

After transfection of SETD7-dCas9 and sgRNA into HCT116, the transfected cells formed spheroids with ADSC and M0 driven in THP-1. Different types of spheroid viability and macrophage spheroid polarization were evaluated. As a result, it was confirmed that SETD7-dCas9 and p300-dCas9 may enhance apoptosis of cancer cells in HCT116, but did not reduce cell viability in the in vitro colorectal cancer microenvironment (FIG. 6A). Furthermore, macrophages showed differences in polarization in 3D colonic spheroids, with a slight decrease in M2 polarization, but epigenetic regulation of HCT116 by SETD7-dCas9 could not enhance the tumor inhibitor phenotype M1 macrophages (FIG. 6B).

### Experimental Example 3: Development of a Predictive Model for SETD7-dCas9 Operation

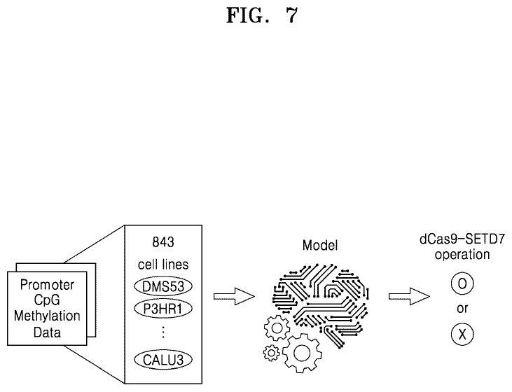

To predict which genes dCas9-SETD7 might act on, DNA methylation data of promoter CpGs from the Cancer Cell Line Encyclopedia (CCLE) in 17 gene promoter clusters of 9 genes (BRD3, EGFL8, HLA-G, TP53, FNTB, ERCC5, VEGFA, CDKN1A, and MDM2) were evaluated (FIG. 7, FIG. 8, and Table 2). Among the analyzed gene promoters, EGFL8 and TP53 showed high DNA methylation levels, which likely contributed to the strong classification performance of the model. This is also consistent with the biological role of SETD7 as a histone methyltransferase, suggesting that such activity may be associated with the epigenetic status of these genes.

A binary classification model of dCas9-SETD7 operation was prepared using the logistic regression (LR) algorithm. A Leave-One-Out Cross-Validation (LOOCV) was performed to evaluate the prediction performance of the model considering a limited number of training instances. Specifically, the LOOCV process involved training a model on all instances except one and then testing the model on the excluded instances. This process was repeated for all instances in turn. To evaluate the generalization function of the model in cross-validation, receiver operating characteristic (ROC) curves and precision-recall (PR) curves were generated, and the area under the curve (AUC) was calculated. After cross-validation, the final model was evaluated by generating a confusion matrix, ROC curve, and PR curve. From these results, it was confirmed that the final model outperformed the cross-validation results. Specifically, the average accuracy of LOOCV was 0.882 (95% CI: 0.706-1.000), including 6 true negatives and 9 true positives. Meanwhile, the accuracy of the final model was 0.941 (95% CI: 0.824-1.000), indicating improved specificity. The ROC AUC of LOOCV was 0.886 (95% CI: 0.652-1.000), and for the final model, it improved to 0.986 (95% CI: 0.917-1.000). Similarly, the PR AUC of LOOCV was 0.947 (95% CI: 0.806-1.000), and the final model showed a significant increase to 0.990 (95% CI: 0.942-1.000).

Furthermore, the best threshold value based on F1-score was evaluated to determine the optimal probability threshold value for operational prediction. During LOOCV, the best threshold value was calculated for each iteration and the average was taken, and after training the final model, another best threshold value was calculated based on the F1-score. The best threshold value of the final model (0.177, F1=0.952) gave a higher F1-score than LOOCV (0.571, F1=0.900), indicating that the threshold value of the final model was more optimal. Since the final model was trained on the entire dataset, this improved threshold value likely represents a more precise decision boundary.

TABLE 2

| | Average DNA methylation value for 843 cell lines | | | | |
|---|---|---|---|---|---|
| Gene | | | | | Applica- |
| Symbol | Mean ± SD | Median | Maximum | Minimum | bility |
| BRD3 | 0.311 ± 0.196 | 0.331 | 0.930 | −0.310 | ◯ |
| EGFL8 | 0.919 ± 0.200 | 1 | 1 | 1 | ◯ |
| HLA-G | 0.643 ± 0.273 | 0.697 | 1.528 | −0.197 | ◯ |
| TP53 | 0.955 ± 0.079 | 0.983 | 1.074 | 0.870 | ◯ |
| FNTB | 0.008 ± 0.026 | 0 | 0 | 0 | X |
| ERCC5 | 0.005 ± 0.009 | 0.002 | 0.015 | −0.009 | X |
| VEGFA | 0.009 ± 0.032 | 0.004 | 0.018 | −0.008 | X |
| CDKN1A | 0.174 ± 0.195 | 0.079 | 0.839 | −0.499 | X |
| MDM2 | 0.040 ± 0.081 | 0.003 | 0.104 | −0.062 | X |

Maximum (Q3 + 1.5 IQR), Minimum (Q1 − 1.5 IQR)

### Experimental Example 4: Validation of Target Gene Up-Regulation by SETD7-dCas9

To evaluate the broad applicability of the SETD7-dCas9 fusion system beyond natural targets, its ability to activate non-natural target genes was analyzed. This analysis is aimed to understand how the system functions when directed by genes not typically associated with SETD7 activity. A OCT4 (POU5F1) was selected as the benchmark target gene of the system, and the MW opsin and RORα genes, which play a role in maintaining vision, were selected to verify the potential of SETD7-dCas9 for therapeutic purposes. To validate the applicability of the target in the SETD7-dCas9 system, the prepared SETD7-dCas9 operation prediction model was first applied to new data including three genes (POU5F1B, OPN1LW, and RORA). The model predicted that dCas9-SETD7 would act on all three genes shown in the heat map visualizing differentially methylated promoter CpG sites of the 12 selected genes (HLA-G, BRD3, EGFL8, TP53, OCT4, OPN1MW, RORA, FNTB, ERCC5, VEGFA, CDKN1A, MDM2) (FIG. 9).

### Experimental Example 5: Confirming Therapeutic Potential of MW Opsin Activation to Compensate for Rhodopsin (RHO) Deficiency in Retinal Disorders

5-1. Rhodopsin Mutation and Compensation Via MW Opsin Expression Using SETD7-dCas9 and p300

A rhodopsin mutation was generated by deleting the E-F loop region between amino acids 231 and 252 using the Gibson assembly method, and these mutations result in reduced conductance activation. A dCas9-rhodopsin EF deletion mutant-GFP plasmid was transfected into HEK293 using Lipofectamine 3000 for 24 hours. An MW opsin sgRNA was then retransfected into cells together with dCas9-SETD7 or dCas9-p300. After 48 hours, the transfected cells were evaluated for light absorbance to confirm the activation of MW opsin due to reduced rhodopsin function. NIH-3T3 cells stably expressing endogenous rhodopsin and HEK293 cells transfected with rhodopsin were used as a control group.

### 5-2. Light Absorption Measurement

Light absorption was measured in the visible spectrum using a UV/Vis spectrometer (Lambda 365+, PerkinElmer, U.S.). Notably, changes in light absorption at a wavelength of 495 nm were monitored, which corresponds to the characteristics of rhodopsin. These characteristics indicate the function of rhodopsin in cells generated by genetic activation by SETD7-dCas9 according to an aspect. The intrinsic light absorbance of the samples was measured without adding 11-cis-retinal to the cells, and the photo-activated samples were checked at various times over time after the addition of 11-cis-retinal.

### 5-3. Experimental Results

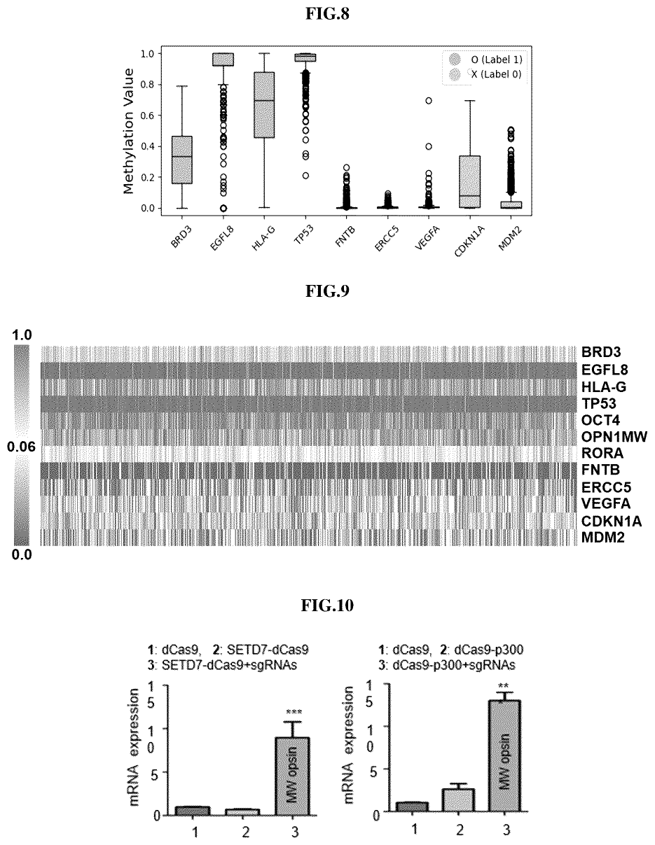

To evaluate the potential of the SETD7-dCas9 system to activate the MW opsin gene essential for photoreceptor function, a series of experiments were performed in the NIH-3T3 cell line, which is known to express low levels of MW opsin. Specifically, NIH-3T3 cells were transfected with SETD7-dCas9 and three gRNAs targeting the TSS region of the MW opsin gene, with the aim of enhancing MW opsin expression. As a positive control group, the dCas9-p300 system, which is well-established for increasing gene expression through histone acetylation, was used. As a result, it was confirmed that co-transfection of SETD7-dCas9 with MW opsin-specific gRNA significantly increased MW opsin expression at both mRNA (FIG. 10) and protein levels (FIG. 11).

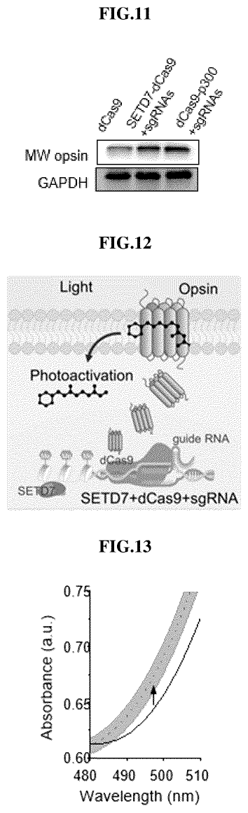

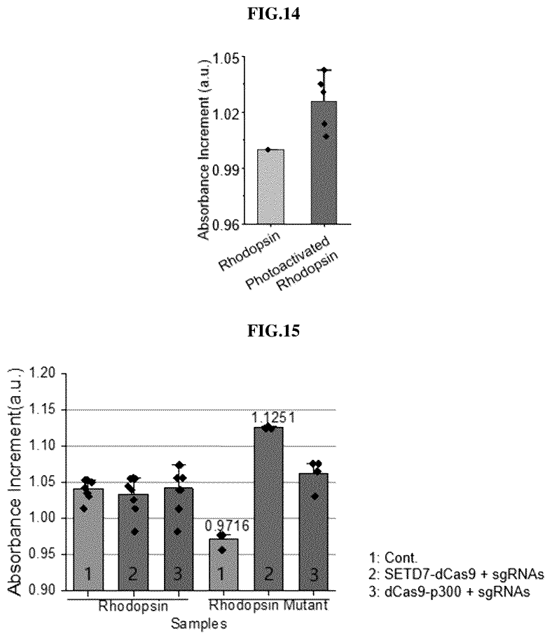

Next, the light absorption property of transfected cells were evaluated to confirm whether the derived MW opsin could mimic the functional property of rhodopsin (RHO). NIH-3T3 cells were treated with 11-cis-retinal to activate the opsin protein, and then the light absorption property of the cells were measured (FIG. 12). As a result, a significant change in light absorption at the characteristic wavelength of RHO was observed after MW opsin up-regulation by SETD7-dCas9, indicating that the MW opsin generated via SETD7-dCas9 activation was functionally active (FIG. 13). Changes in the absorption spectrum of NIH-3T3 cells expressing rhodopsin also indicate increased light absorption. Control experiments without 11-cis-retinal treatment or light exposure showed no significant changes in light absorption, indicating that the observed changes in light absorption are specific to activated MW opsin or rhodopsin (FIG. 14). These results indicate that the SETD7-dCas9 system not only up-regulates MW opsin expression but also promotes functional opsin activity, reflecting the spectral behavior of RHO.

In addition, as a result of comparative analysis of the light absorbance in cells expressing WT rhodopsin, cells with rhodopsin mutations, and cells with and without MW opsin up-regulation by the SETD7-dCas9 and dCas9-p300 systems, it was confirmed that MW opsin has the potential to compensate for rhodopsin deficiency (FIG. 15).

The disclosure relates to a fusion protein including a transcription enhancer and a dCas9 protein, a complex for epigenetic regulation including the fusion protein and one or more guide RNAs (gRNAs), etc., wherein the fusion protein or complex may induce the apoptosis of colorectal cancer cells or compensate for rhodopsin deficiency by activating genes such as EGFL8, opsin mw, etc., and may be utilized as a pharmaceutical composition, etc., for preventing or treating cancer or ocular diseases.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

1. A fusion protein comprising a transcription enhancer and an inactivated Cas9 (dCas9) protein,
wherein the transcription enhancer is SETD7 or a variant thereof.

2. The fusion protein of claim 1, wherein the transcription enhancer and dCas9 are linked via a linker.

3. The fusion protein of claim 2, wherein the linker consists of 10 to 15 amino acids in length.

4. The fusion protein of claim 1, further comprising a nuclear localization signal (NLS) peptide.

5. A polynucleotide encoding the fusion protein of claim 1.

6. A vector comprising the polynucleotide of claim 5.

7. A complex for epigenetic regulation, comprising: a fusion protein comprising a transcription enhancer and an inactivated Cas9 (dCas9) protein; and one or more guide RNAs (gRNAs),
wherein the transcription enhancer is SETD7 or a variant thereof.

8. A composition for gene regulation, comprising the complex of claim 7.

9. The composition of claim 8, wherein the gene is any one or more selected from the group consisting of EGFL8, HLA-G, BRD3, FNTB ERCC5, Opsin mw, STK33, and TNIK.
